(19) United States
(12) Patent Application Publication
(10) Pub. No.: US 2023/0230691 A1
(43) Pub. Date: Jul. 20, 2023

(54) WEARABLE DEVICE TRAVEL EMERGENCY HEALTH ASSISTANCE

(71) Applicant: International Business Machines Corporation, Armonk, NY (US)

(72) Inventors: Manish Anand Bhide, Hyderabad (IN); Madan K Chukka, Hyderabad (IN); Phani Kumar V. U. Ayyagari, Hyderabad (IN); Sai Prasanth Vuppala, Visakhapatnam (IN); Purnachandra Rao Jasti, Hyderabad (IN)

(21) Appl. No.: 17/575,748

(22) Filed: Jan. 14, 2022

(51) Int. Cl. G16H 40/67; G06F 1/16; G06Q 10/02

(52) U.S. Cl. CPC G16H 40/67; G06F 1/163; G06Q 10/02

(57) ABSTRACT

A health status of a first user is monitored from a wearable device. The health status is based on a set of one or more health metrics captured by the wearable device. The health status is measured while the wearable device is worn by the first user. It is determined that the first user is participating in a travel event that is related to a travel vehicle. A potential health concern of the first user is detected based on the health status. An intervention action related to the health status is performed, in response to the potential health concern. The intervention action is performed based on the travel event.

### BACKGROUND

The present disclosure relates to wearable computing, and more specifically, to performing assistance actions in response to wearable device states.

Wearable devices may be computers that are worn by a user. Wearable devices may be configured to notify certain individuals regarding the health of a wearer. The wearable devices may rely on a communication network, such as the Internet, to perform notifications.

### SUMMARY

According to embodiments, disclosed are a method, system, and computer program product.

A health status of a first user is monitored from a wearable device. The health status is based on a set of one or more health metrics captured by the wearable device. The health status is measured while the wearable device is worn by the first user. It is determined that the first user is participating in a travel event that is related to a travel vehicle. A potential health concern of the first user is detected based on the health status. An intervention action related to the health status is performed, in response to the potential health concern. The intervention action is performed based on the travel event.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts the representative major components of an example computer system that may be used, in accordance with some embodiments of the present disclosure;

FIG. 2 depicts a system configured to perform wearable assistance during travel, consistent with some embodiments of the disclosure; and

FIG. 3 Depicts an example method of perform assistance to users, consistent with some embodiments of the disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to wearable computing; more particular aspects relate to performing assistance actions in response to wearable device states. While the present disclosure is not necessarily limited to such applications, various aspects of the disclosure may be appreciated through a discussion of various examples using this context.

Wearable comping is a rapidly advancing segment of technology. Specifically, wearable devices may be computers that are worn by a user, such as watches and fitness bands. With the advancement of processing power, such as integrated circuits, wearable devices are becoming cheaper and are more commonly used in every-day scenarios. For instance, many users can readily afford and may chose to wear a watch everyday that includes computing elements, such as processor, memory, and input/output (e.g., a smart watch, a fitness band, a wearable computer).

Wearable devices may be configured to monitor or identify the health of a user. For example, a smart watch may be configured to measure a blood oxygen level of the wearing user (“user”). In another example, a fitness band may be worn by a user, and may be configured to monitor the heart-rate of a user. The wearable devices may record or communicate the health measurements. For example, wearable devices may include a wireless communication transceiver, such as a Wi-Fi antenna. The wearable devices may communicate the information to other users through the communication transceiver. For example, a smart watch may be configured to notify a doctor of the wearing user.

Wearable devices may be limited in their ability to be useful in all situations. The wearable devices may have deficiencies in how to communicate an emergent situation. Specifically, a wearable device may rely on a communication network, such as the Internet, to perform notifications. If the wearable device is located outside of network communication, the ability to monitor vitals of a user may be of limited value. For example, if a user is on a subway, in the air, or at sea, a wearable device may fail to notify anybody of a problem because it does not have a network connection. While traveling on certain travel vehicles (e.g., high speed rail, airplanes, transcontinental ships), a user may have no network connection, and may have limited ability to deal with health concerns. For example, if a user is suffering from a breathing issue while on an airplane flight, the user's wearable device may be ineffective by attempting to wirelessly communicate outside of the flight.

Further limitations may be in the configuration of who to notify. Specifically, wearable devices may be configured to notify certain individuals regarding the health of a wearer. These individuals may be fixed regardless of where a user is. For example, a user may set an emergency contact that is a roommate. When the user is in their dwelling or in their neighborhood, the user may suffer from an ailment, and the wearable device may be configured to monitor and identify the ailment. In response to identifying the ailment, the user's wearable device may notify the roommate who can get help for the user. Unfortunately, this fixed notification may be of little value while the user is traveling. For instance, a notification to a roommate when the user is traveling may be of little value, because the roommate may not be able to do anything to help the user.

Travel Aware Wearable Assistance (“TAWA”) may operate to aid a user that wears a wearable device. The assistance provided by TAWA may operate in instances that are more appropriate for a traveling user. TAWA may include a wearable device that is configured to monitor a health status (“health”) of a user. The health status may include one or more health metrics monitored while the wearable device is worn by the user. TAWA may include one or more devices performing a detection of a potential health concern. Specifically, TAWA may operate on one or more client devices (e.g., one or more wearable devices, on a paired mobile device of a user) configured to detect a change in health metrics of a user that indicates a potential health concern. For example, a wearable device may be configured to detect a change in heart rate or oxygen level and determine that the change may correlate with a dangerous or serious health condition (“potential health condition”) of the wearing user. TAWA may be configured to perform an intervention action, such as notifying a designated user. For example, a user may configure a wearable device, in response to detecting potential health conditions, to notify a close relative, a friend, or roommate.

TAWA may overcome the various limitations of existing wearable devices. Specifically, TAWA may operate to alter or update the manner of an intervention action based on a state of a user. Further, TAWA may also operate to alter the target user of a communication that is part of an intervention action. For instance, TAWA may be configured to determine whether the user that is wearing a wearable device while the user is traveling. For instance, a travel event may be a trip that the user is taking (or is planning to, beginning to take). TAWA may be configured to identify actions while the user is taking the trip, such as reaching a location related to a travel event (e.g., a terminal) or entering a travel vehicle related to the travel event. Responsive to determining the user is active or participating in the travel event, TAWA may include reconfiguring the intervention action. For example, TAWA may include transmitting notification or emergency alerts that are initiated from a wearable device to a crew member of the travel vehicle. In another example, TAWA may include a wearable device initiating a health or safety measure, such as deploying an oxygen mask. Travel companies may be configured to provide TAWA optionally. For example, an airline may offer TAWA as an optional service, upgraded service, emergency service, or accessibility service, similar to wheelchair availability. TAWA may be offered during the ticket booking, or upon arrival at a terminal.

To overcome various limitations of existing wearables, the TAWA may operate while being resilient to intermittent or changing network access or a missing network connection. Specifically, other wearable or client devices may attempt, without success, to communicate without a network connection while on a trip or travel event; conversely, TAWA may operate successfully to get help for a user even without any network access. Operation without network access may include a wearable or wearable device operating with no Internet access, operating with no network connection, operating without establishing a network connection, operating while in airplane mode, operating when a network connection is lost, operating after a network connection is disconnected, and/or otherwise operating without a network. Operation without network access may include a wearable device successfully communicating to another entity as part of an intervention action when the wearable device does not have any communicative connection (e.g., network access) to one or more of the emergency contacts or designated emergency contacts. TAWA may be ideal in situations related to travel events that do not have a network. Specifically, some travel vehicles may not have a network connection, such as an airplane, train, or ship without Internet access. TAWA may utilize local communication networks that are a part of the travel vehicle to communicate without a client device of the user having a connection to other networks. TAWA may also create an ad-hoc network between devices within a personal area network range to indicate a need for an intervention action.

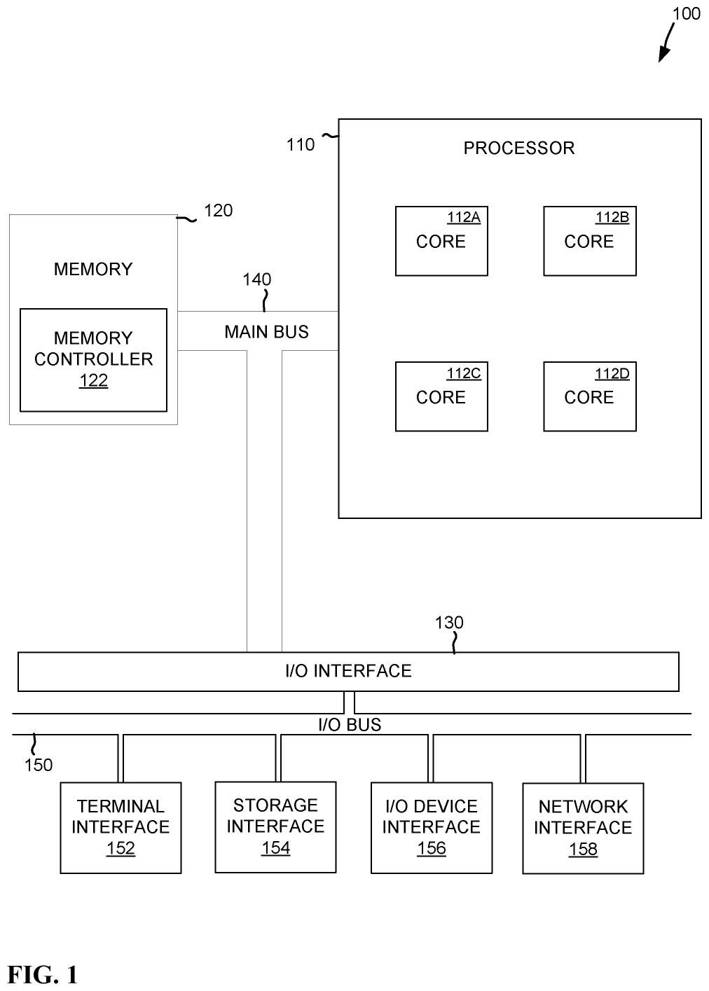

FIG. 1 depicts the representative major components of an example computer system 100 (alternatively, computer) that may be used, in accordance with some embodiments of the present disclosure. It is appreciated that individual components may vary in complexity, number, type, and/or configuration. The particular examples disclosed are for example purposes only and are not necessarily the only such variations. The computer system 100 may include a processor 110, memory 120, an input/output interface (herein, I/O or I/O interface) 130, and a main bus 140. The main bus 140 may provide communication pathways for the other components of the computer system 100. In some embodiments, the main bus 140 may connect to other components such as a specialized digital signal processor (not depicted).

The processor 110 of the computer system 100 may be comprised of one or more cores 112A, 112B, 112C, 112D (collectively 112). The processor 110 may additionally include one or more memory buffers or caches (not depicted) that provide temporary storage of instructions and data for the cores 112. The cores 112 may perform instructions on input provided from the caches or from the memory 120 and output the result to caches or the memory. The cores 112 may be comprised of one or more circuits configured to perform one or more methods consistent with embodiments of the present disclosure. In some embodiments, the computer system 100 may contain multiple processors 110. In some embodiments, the computer system 100 may be a single processor 110 with a singular core 112.

The memory 120 of the computer system 100 may include a memory controller 122. In some embodiments, the memory 120 may include a random-access semiconductor memory, storage device, or storage medium (either volatile or non-volatile) for storing data and programs. In some embodiments, the memory may be in the form of modules (e.g., dual in-line memory modules). The memory controller 122 may communicate with the processor 110, facilitating storage and retrieval of information in the memory 120. The memory controller 122 may communicate with the I/O interface 130, facilitating storage and retrieval of input or output in the memory 120.

The I/O interface 130 may include an I/O bus 150, a terminal interface 152, a storage interface 154, an I/O device interface 156, and a network interface 158. The I/O interface 130 may connect the main bus 140 to the I/O bus 150. The I/O interface 130 may direct instructions and data from the processor 110 and memory 120 to the various interfaces of the I/O bus 150. The I/O interface 130 may also direct instructions and data from the various interfaces of the I/O bus 150 to the processor 110 and memory 120. The various interfaces may include the terminal interface 152, the storage interface 154, the I/O device interface 156, and the network interface 158. In some embodiments, the various interfaces may include a subset of the aforementioned interfaces (e.g., an embedded computer system in an industrial application may not include the terminal interface 152 and the storage interface 154).

Logic modules throughout the computer system 100—including but not limited to the memory 120, the processor 110, and the I/O interface 130—may communicate failures and changes to one or more components to a hypervisor or operating system (not depicted). The hypervisor or the operating system may allocate the various resources available in the computer system 100 and track the location of data in memory 120 and of processes assigned to various cores 112. In embodiments that combine or rearrange elements, aspects and capabilities of the logic modules may be combined or redistributed. These variations would be apparent to one skilled in the art.

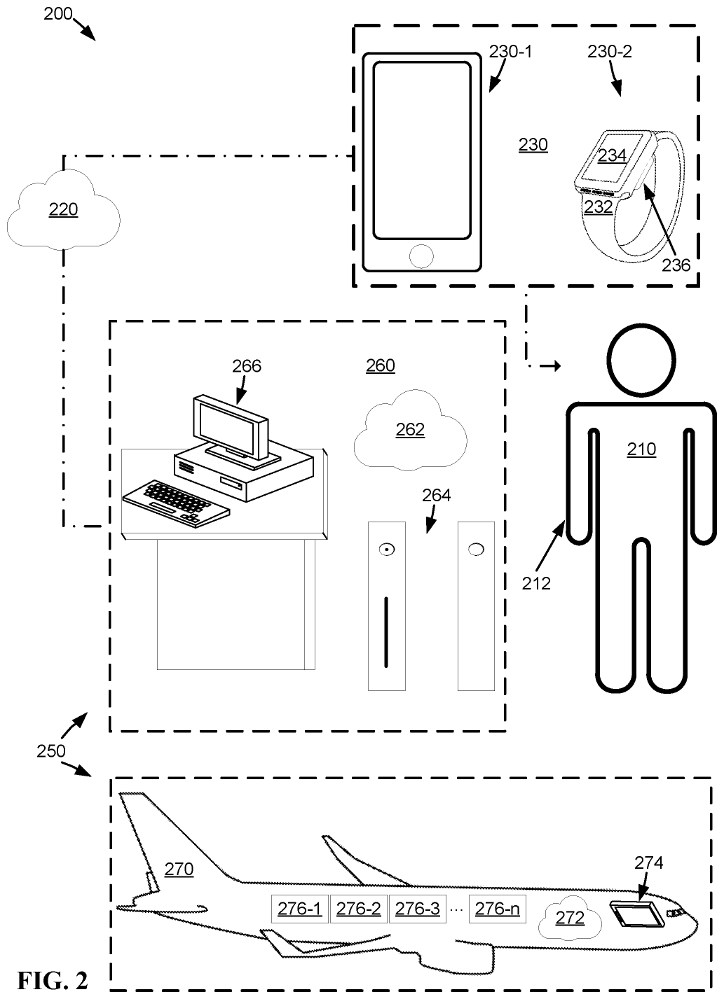

FIG. 2 depicts a system 200 configured to perform wearable assistance during travel, consistent with some embodiments of the disclosure. Specifically, system 200 may be configured to perform one or more operations of TAWA for a user 210. System 200 may include one or more of the following: a general network 220 and a set of one or more client devices 230 of the user 210.

General network 220 can be implemented using any number of any suitable physical and/or logical communications topologies. The general network 220 can include one or more private or public computing networks. For example, general network 220 may comprise a private network (e.g., a network with a firewall that blocks non-authorized external access) that is associated with a particular function or workload (e.g., communication, streaming, hosting, sharing), or set of software or hardware clients. Alternatively, or additionally, general network 220 may comprise a public network, such as the Internet. Consequently, general network 220 may form part of a data unit network (e.g., packet-based)—for instance, a local-area network, a wide-area network, and/or a global network.

General network 220 can include one or more servers, networks, or databases, and can use one or more communication protocols to transfer data between other components of system 200. Furthermore, although illustrated in FIG. 2 as a single entity, in other examples, general network 220 may comprise a plurality of networks, such as a combination of public and/or private networks. General network 220 can include a variety of types of physical communication channels or “links.” The links can be wired, wireless, optical, and/or any other suitable media. In addition, general network 220 can include a variety of network hardware and software (not depicted) for performing routing, switching, and other functions, such as routers, switches, base stations, bridges or any other equipment that may be useful to facilitate communicating data.

The set of client devices 230 may include a first client device 230-1 and a second client device 230-2. The client devices 230 may be computing devices, such as a laptop computer, a tablet computer, a smartphone, or the like. Specifically, each of the client devices 230 may be a computer system, such as computer 100. Each client device 230 may include a processor and memory, and may be configured to execute operations to perform TAWA. Client device 230-1 may be a tablet or smartphone carried by a user as they navigate real-world environments. For example, client device 230-1 may be a smartphone that is carried, such as in a pocket, by user 210. Client device 230-1 may be configured to receive input and present output to users. For example, client device 230-1 may be configured to receive touchscreen or verbal input from user 210 and may, responsive to input, provide information to the user such as visual and auditory output.

Each client device 230 may include a network interface (not depicted) configured to communicate with other computers. Specifically, client device 230-1 may include a transceiver (not depicted) for communication to various computer networks. For example, client device 230-1 may be configured to broadcast availability to (and communicate with) various networks while it translates through real-world environments. In another example, client device 230-1 may be configured to connect to general network 220, to communicate with other users. Similarly, client device 230-2 may include a transceiver (not depicted) for communication to various computer networks. For example, client device 230-2 may be configured to broadcast availability to (and communicate with) various networks while it translates through real-world environments, such as general network 220. The client devices 230 may be configured to communicate with each other indirectly, such as through networks. For example, client device 230-1 may communicate through general network 220 to communicate with client device 230-2. The client devices 230 may be configured to communicate with each other directly, such as through an ad-hoc network. For example, client device 230-2 may directly, without communicating to another network, establish a communication channel with client device 230-1.

One or more of the client devices 230 may be a wearable device, such as a smart watch, health band, fitness band, and/or health monitor. For example, as depicted in FIG. 2, user 210 may wear client device 230-2 on their right wrist 212. Client device 230-2, may include at least the following: a band 232, a display 234, and health sensor 236. As depicted in FIG. 2, user 210 may wear client device 230-2 on their right wrist 212. The band 232 may be a fabric, rubber, plastic, or metal loop that enables client device 230-2 to be worn on a leg, arm, or around the shoulders of a user. The display 234 may be a liquid crystal or organic light emitting display configured to render output of processing to a user. For example, client device 230-2 may present a read-out of various information through display 234, for user 210 to view and/or read, such as text messages, visual statuses, and/or health information.

The client devices 230 may be configured to monitor a health status of a user. For example, client device 230-2 may utilize health sensor 236 to monitor one or more health metrics on/through the skin of user 210. Specifically, health sensor 236 may be glass, ceramic, sapphire, or another relevant surface that allows for the passage of sound waves and various spectrums of light. The health sensor 236 may be configured to monitor one or more metrics that relate to the oxygen of a user, such as the amount of oxygen in the blood of a user at a particular point (e.g., at wrist 212 of user 210). The health sensor 236 may be configured to monitor one or more metrics that relate to the blood of a user, such as the rate of blood that moves through the user at a particular point. The health metrics captured by health sensor 236 may be used to monitor a health status of a user. The health status may be based on a combination of the blood and oxygen measured from the user, and to derive information of a user. For example, the client device 230-2 and/or the client device 230-1 may utilize the health metrics captured by the health sensor to determine one or more of the following of a user: a pulse, a heart rate, an electrocardiogram, a blood glucose level, a blood pressure, a respiratory rate, and/or an oxygen saturation.

While moving through real-world environments, the client devices 230 may operate to communicate a health status to a relevant party. For example, user 210 may carry with them or wear client devices 230. If a potential health concern is detected, such as through health measurements captured by client device 230-2, one or both of the client devices 230 may notify user 210 (e.g., a beep sound, a visual alert, or other relevant notification). Additionally, or alternatively, if a potential health concern is detected, a relevant client device 230 may notify another user. For example, user 210 may designate, through a software application, or system setting, one or more relevant personal contacts as emergency contacts. In another example, client device 230 may identify relevant designated emergency contacts, such as local emergency police or firefighters. If a potential health concern is detected, the relevant client device 230 may communicate with the emergency contact, such as by sending them a message related to the health status. For example, a parent or guardian of user 210 may be notified if there is a change in a blood measurement or other relevant health metric that indicates a potential health situation (e.g., atrial fibrillation, ventricular tachycardia, relatively high blood pressure).

System 300 may also operate by performing TAWA during relevant times and places. Specifically, system 300 may operate during a travel event 250. Travel event 250 may be related to a particular type of trip that is far away from a normal residence of a user or is during a situation where a user may not have normal contact with other users, such as those designated as emergency contacts. Travel event 250 may include the following: a travel location 260 and a travel vehicle 270. For purposes of example, travel location 260 may be for a flight on a travel vehicle 270 that is an airplane. Specifically, travel location 260 may be an international airline terminal for arrivals and boarding of jets and other planes, and travel vehicle 270 may be a jet airplane related to a commercial airline flight between countries. Though FIG. 2 depicts an airline trip including an airline terminal 260 and an airplane 270, other scenarios of TAWA may be contemplated by system 300 (e.g., a port and international ship for boat travel, a train station, and a train for railway travel).

Travel location 260 may be a terminal, station, or other designated location that is related to a travel event 250. Travel location 260 may include the following, a location network 262; a location ticket reader 264; and at least one location computer 266. Location network 262 may be an instance of a communications network, including relevant hardware and software, such as switches and routers. Location network 262 may be communicatively coupled to other networks, such as general network 220. Location network 262 may broadcast or advertise a specific set of information that defines or identifies an association with the travel location 260. For example, a wireless access point name, relevant SSID, MAC address, global position system (“GPS”) information of location network 262 may uniquely identify travel location 260. Location ticket reader 264 may be a specialized hardware reader that is configured to read one or more boarding passes of a user, such as user 210. For example, location ticket reader 264 may be a paper ticket reader that may be configured to read human-readable or machine-readable visual information on a paper ticket. In another example, location ticket reader 264 may be an electronic reader configured with near field communication, a personal area network, or other relevant technology capable of communicating with a client device 230 of user 210 to read a digital ticket. Location computer 266 may represent one or more computer systems that are at travel location 260. Location computer 266 may be a ticket terminal, boarding computer, or other device where a user may purchase a travel ticket or check-in to a trip.

In performing TAWA, system 200 may react to the presence of travel location 260 and may determine that a user is participating in a travel event 250. Specifically, user 210 may be approaching the travel location 260 in order to board for an international flight back home. Various components of system 200 may then react to identify the travel location 260, and consequently determine the user is participating in the travel event 250. For example, as the user approaches the travel location 260, travel network 262 may broadcast network signals to any devices in the proximity and client devices 230 may identify the travel network 262. In another example, as the user swipes client device 230-1, a digital ticket in a digital wallet application on client device 230-1 may be confirmed by location ticket reader 264. In yet another example, based on GPS sensors in a client device 230, it may be determined the user is participating in the travel event 250. In another example, based on a user performing a boarding operation with one or more computers, such as location computer 266 either alone or in conjunction with the client devices 230, it may be identified that the user is at the travel location 260.

The elements of system 200 may operate to perform intervention actions related to a health status of user 210. Specifically, in determining that the user is participating in the travel event, and based on monitoring by the client devices 230, a potential health concern of user 210 may be detected. For example, while the user is at travel location 260, the user may be suffering from a change in heart rate above or below a threshold rate. The system 300 may be configured to perform an intervention action in such situation. For example, a notification may be sent by one or more of the client devices 230 to a staff member of the travel location 260. In another example, an alert may be displayed by location computer 266 to an attendant that works in a region of the travel location 260 near the user. The notification to an attendant or other individual located at the travel location 260 may be performed by system 200, despite one or more of the client devices 230 having network connectivity to general network 220. For example, TAWA may be performed by a component of system 300, despite a client device 230 having communicative capability with a designated or default emergency contact of user 210.

Travel vehicle 270 may be a vehicle that is related to travel event 250. Travel vehicle 270 may include the following: a vehicle network 272; a vehicle communicator 274; and one or more passenger devices 276-1, 276-2, 276-3, up to 276-n (collectively, passenger devices 276). The vehicle network 272 may be a passenger accessible network that is provided to passengers of the travel vehicle 270. Specifically, vehicle network 272 may be one or more wired or wireless access points and associated media servers. The vehicle network 272 may communicatively couple client devices of passengers, such as client devices 230, to media servers contained on travel vehicle 270. For example, vehicle network 272 may facilitate the playing of movies, music for passengers to consume on their client devices. In another example, vehicle network 272 may include a local area network that permits client devices of users to host chat and games (e.g., multi-user chatrooms, multi-player games). The vehicle network 272 may or may not be communicatively coupled to other networks, such as general network 220. As a result, during travel, travel vehicle 270 may not permit client devices of users, such as client devices 230, to communicatively couple to any entity that is outside of travel vehicle 270.

Vehicle communicator 274 of travel vehicle 270 may include one or more radios, displays, or other status screens, that may communicate information to one or more crew members (not depicted) of the travel vehicle 270. Vehicle communicator 274 may represent a network reserved for communication between crew members of the travel vehicle 270. Passenger devices 276 may include one or more devices installed in travel vehicle 270 for use and benefit of passengers. For example, passenger devices 276 may include one or more of the following: call attendant buttons, oxygen masks, overhead lights, screens, seat belts, and/or alarms. The passenger devices 276 may be associated with the number of passengers. For example, if travel vehicle 270 is configured to house one-hundred eighty passengers, then there may be one-hundred eighty passenger devices 276.

In performing TAWA, system 200 may react to the presence of travel vehicle 270 and may determine that a user is participating in a travel event 250. Specifically, user 210 may be in or on the travel vehicle 270 in order to fly to a destination. Various components of system 200 may then react to identify the travel vehicle 270, and consequently determine the user is participating in the travel event 250. For example, as the user approaches the travel vehicle 270, vehicle network 272 may broadcast network signals to any devices in the proximity and client devices 230 may identify the vehicle network 272. In another example, as the user consumes media or interacts with client devices, vehicle network 272 may detect the presence of a user. In yet another example, based on GPS sensors in a client device 230, it may be determined the user is traveling on the travel vehicle 270. In another example, based on a user performing a boarding operation with one or more computers, such as location computer 266, either alone or in conjunction with the client devices 230, it may be identified that the user is on the travel vehicle 270.

The elements of system 200 may operate to perform intervention actions related to a health status of user 210. Specifically, in determining that the user is participating in the travel event 250, and based on monitoring by the client devices 230, a potential health concern of user 210 may be detected. For example, while the user is traveling on travel vehicle 270, the user may be suffering from a change in oxygen saturation. The system 300 may be configured to perform an intervention action in such situations that includes a notification. For example, a notification may be sent by one or more of the client devices 230 to a crew member of the travel vehicle 270. The notification may be directly sent to travel communicator 274. For example, client device 230-2 may be communicatively coupled to travel computer 274 in response to a user boarding the travel vehicle 270. The notification may be indirectly sent to travel computer 274. For example, client device 230-2 may communicate to client device 230-1, and client device 230-1 may communicate to travel computer 274. In another example, client device 230-1 may communicate through passenger network 272 to vehicle communicator 274. In yet another example, a pilot or other crew member may be instructed to provide assistance or to seek an emergency landing as soon as possible, in response to the notification.

Communication through the travel vehicle may also use one or more ad-hoc or device to device communication technologies, such as a personal area network (e.g., Bluetooth). For example, client device 230-1 may communicate with other client devices of other passengers (not depicted), and the other client devices may communicate to vehicle communicator 274. Other passengers (not depicted) may be able to opt-in to assisting or receiving notifications as part of the intervention actions. For example, a passenger that is a doctor may opt-in to receiving notifications about potential health conditions of other passengers. Responsive to a potential health condition, client device 230-1 may communicate or send a notification (e.g., directly through an ad-hoc network, through passenger network 272) to the passenger that is a doctor on the travel vehicle 270. The notification to another individual (e.g., a crew member, an attendant, another user) inside of the travel vehicle 270 may be performed by system 200, despite one or more of the client devices 230 having or not having network connectivity to general network 220. For example, TAWA may be performed by a component of system 300, despite a client device 230 having or not having communicative capability with a designated or default emergency contact of user 210.

The system 200 may be configured to perform an intervention action in situations of a detected potential health concern other than a notification. Specifically, user 210 may be seated at a passenger seat that is associated with passenger device 276-2 on travel vehicle 270. In response to detecting the potential health concern by client device 230-2, client device 230-2 may instruct passenger device 276-2 to activate. For example, passenger device 276-2 may be a deployable oxygen mask that is configured to deploy responsive to being instructed to activate. In another example, passenger device 276-2 may be an attendant call button and associated light and noise (e.g., tone, ring, buzz, chime), and in response to being instructed to activate, passenger device 276-2 may begin to illuminate and/or emit sound.

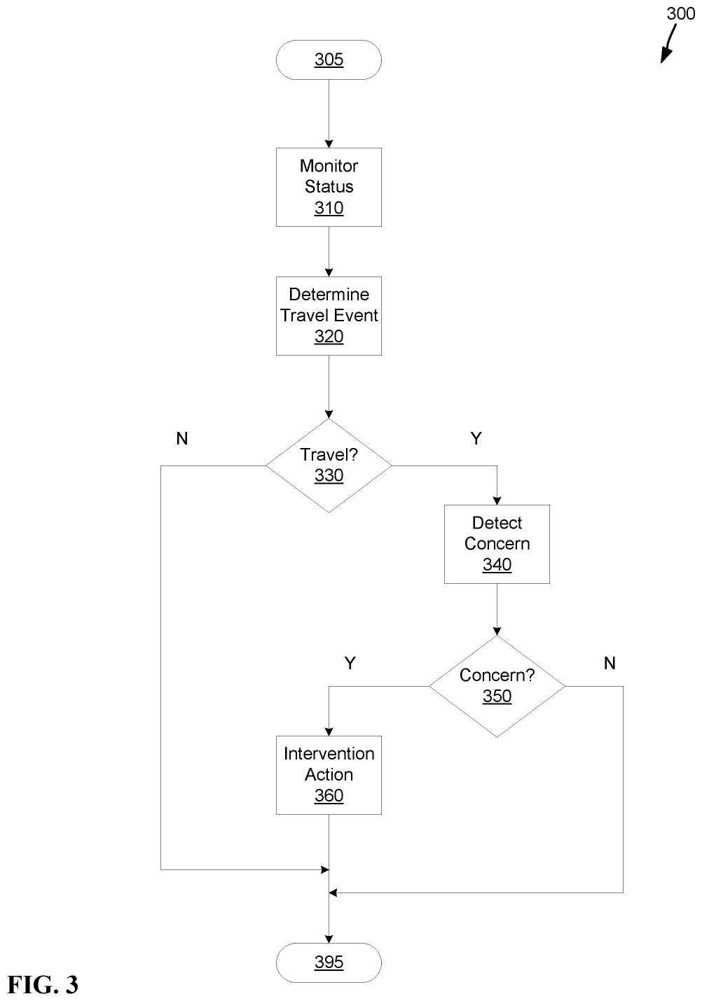

FIG. 3 depicts an example method 300 of performing user assistance, consistent with some embodiments of the disclosure. Specifically, method 300 may include operations of TAWA, such as by performing one or more steps of components of system 200. Method 300 may generally be implemented in fixed-functionality hardware, configurable logic, logic instructions, etc., or any combination thereof. For example, the logic instructions might include assembler instructions, ISA instructions, machine instructions, machine dependent instructions, microcode, state-setting data, configuration data for integrated circuitry, state information that personalizes electronic circuitry and/or other structural components that are native to hardware (e.g., host processor, central processing unit/CPU, microcontroller, etc.).

From start 305, method 300 may begin by monitoring a health status of a user at 310. Monitoring of a health status of a first user may be performed by a wearable device that is worn by a user, such as a smart watch or health band. For example, client device 230-2 may monitor the health status of a user while the client device 230-2 is worn by the user. The health status may be based on a set of one or more health metrics captured by the wearable device. For example, health metrics may include oxygen measurements of a user. In another example, health metrics may include cardiac measurements of a user.

At 320, it may be determined that the user is participating in a travel event. The travel event may be related to a travel vehicle, such as a boat or plane. The determination of the travel event may be performed by a client device (e.g., a wearable device, client device 230-2). The determination of the travel event may be performed indirectly, such as by a communicatively coupled client device that is in communication with another computing device (e.g., client device 230-1 communicating the participation to client device 230-2, client device 230-2 communicating the participation to client device 230-1, location computer 266 communicating the participation to client device 230-2). The determination may be based on a sensor, such as a GPS sensor that is contained in a client device. The determination may be based on a particular travel-related action. For example, a computing device may detect or determine that a digital boarding pass or paper boarding pass was scanned to authenticate a user to travel. In another example, a computing device may detect a proximity to a travel location and/or travel vehicle.

If a user is determined to be participating in a travel event at 330:Y, a potential health concern may be detected at 340. The potential health concern may be detected based on the health status measured by a wearable device. One or more health metrics captured by the wearable device may indicate a change in a health status of the wearing user that is a potential health concern. For example, a drop or rise in a blood measurement may indicate an issue with one of the vital organs of the user, such as the heart. In another example, a change in an oxygen measurement may indicate an issue with a user, such a below-threshold amount of oxygen in the blood.

If a potential health concern is detected at 350:Y, an intervention action may be performed at 360. The intervention action includes activating a safety system of the travel vehicle (e.g., enabling a fasten seatbelt sign, turning on/off cabin lights, deploying an oxygen mask, turning on the call attendant alarm). The intervention action may include communication or transmission of a message or notice to a relevant individual related to the travel event. For example, while a user is traveling on an airplane, a flight attendant may be notified. The notification may include information about the user, such as the potential health concern. The notification may be based on another notification. For example, the wearable device may be configured to generate an emergency contact notification that is directed to a designated emergency contact, such as a friend or relative.

At 360, performing of the intervention action may include intercepting the emergency contact notification and generating an emergency travel notification that includes one or more of the details of the emergency contact notification. In some embodiments, only a subset of the detail of the emergency contact notification may be used to generate the emergency travel notification. For example, an emergency travel notification that is to be broadcast to a flight attendant may not include a last name or other potentially sensitive information that was originally a part of the emergency contact notification. The intervention action may include the rerouting or disabling of communications to other individuals, such as emergency contacts. The intervention action may include delaying of communication to emergency contacts. For example, a wearable device may be configured to generate an emergency contact notification to a relative of a user wearing the wearable device. During the travel event, the emergency contact notification may be intercepted by an airplane and an emergency travel notification may be created and sent to crew members. At a preset delay or period of time (e.g., five minutes, one hour), the emergency contact notification may be sent by the airplane to the emergency contact.

After the intervention action is performed at 360 (or if there is no travel event detected at 330:N or there is no potential health concern detected at 350:N), method 300 may end at 395.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein. cm What is claimed is:

1. A method comprising:
monitoring a health status of a first user from a wearable device,
wherein the health status is based on a set of one or more health metrics captured by the wearable device, and wherein the health status is measured while the wearable device is worn by the first user;
determining that the first user is participating in a travel event that is related to a travel vehicle;
detecting, based on the health status, a potential health concern of the first user; and
performing, in response to the potential health concern and based on the travel event, an intervention action related to the health status.

2. The method of claim 1, wherein the wearable device is a watch.

3. The method of claim 1, wherein the wearable device is a health band.

4. The method of claim 1, wherein the health metrics are selected from the group consisting of an oxygen measurement of the first user, and a blood measurement of the first user.

5. The method of claim 1, wherein
the travel vehicle is an airplane, and
the travel event is the first user is on the airplane for an airline flight.

6. The method of claim 1, wherein
the travel vehicle is a train, and
the travel event is the first user is on the train for a railway trip.

7. The method of claim 1, wherein the wearable device is communicatively coupled to a client device of the first user.

8. The method of claim 7, wherein the travel event is based on a global position system sensor of the client device.

9. The method of claim 7, wherein the travel event is based on a communication signal of the client device.

10. The method of claim 9, wherein
the client device includes a digital boarding pass, and
the determining that the first user is participating in the travel event includes determining
that the digital boarding pass was scanned by the client device.

11. The method of claim 1, wherein the determining that the first user is participating in the travel event further comprises:
detecting a computing device located proximate to the travel event.

12. The method of claim 11, wherein the computing device is located at a terminal that is related to the travel event.

13. The method of claim 11, wherein the computing device is located in the travel vehicle.

14. The method of claim 1, wherein the intervention action includes activating a safety system of the travel vehicle.

15. The method of claim 1, wherein the intervention action includes transmitting an emergency travel notification that is directed to a crew member of the travel vehicle.

16. The method of claim 14, wherein the wearable device is configured to generate an emergency contact notification that is directed to a designated emergency contact, and wherein designated emergency contact is a second user designated by the first user, and wherein the method further comprises:
intercepting the emergency contact notification; and
generating, based on the emergency contact notification, the emergency travel notification.

17. A system, the system comprising:
a memory, the memory containing one or more instructions; and
a processor, the processor communicatively coupled to the memory, the processor, in response to reading the one or more instructions, configured to:
monitor a health status of a first user from a wearable device,
wherein the health status is based on a set of one or more health metrics captured by the wearable device, and wherein the health status is measured while the wearable device is worn by the first user;
determine that the first user is participating in a travel event that is related to a travel vehicle;
detect, based on the health status, a potential health concern of the first user; and
perform, in response to the potential health concern and based on the travel event, an intervention action related to the health status.

18. The system of claim 17, wherein the wearable device is a watch.

19. A computer program product, the computer program product comprising:
one or more computer readable storage media; and
program instructions collectively stored on the one or more computer readable storage media, the program instructions configured to:
monitor a health status of a first user from a wearable device,
wherein the health status is based on a set of one or more health metrics captured by the wearable device, and wherein the health status is measured while the wearable device is worn by the first user;
determine that the first user is participating in a travel event that is related to a travel vehicle;
detect, based on the health status, a potential health concern of the first user; and
perform, in response to the potential health concern and based on the travel event, an intervention action related to the health status.

20. The computer program product of claim 19, wherein the wearable device is a watch.
